(19) United States
(12) Patent Application Publication
(10) Pub. No.: US 2007/0225316 A1
(43) Pub. Date: Sep. 27, 2007

(54) Methods and compositions for treating schizophrenia

(76) Inventors: Sergei O. Bachurin, Chernogolovka (RU); Vladimir V. Grigoriev, Chernogolovka (RU); Margarita A. Morozova, Chernogolovka (RU); Allan G. Beniashvili, Chernogolovka (RU)

Correspondence Address: MORRISON & FOERSTER LLP, 755 PAGE MILL RD, PALO ALTO, CA, 94304-1018, US

(21) Appl. No.: 11/698,318

(22) Filed: Jan. 25, 2007

(30) Foreign Application Priority Data
Jan. 25, 2006 (RU) 2006101999

(51) Int. Cl. A61K 31/4745

(52) U.S. Cl. 514/291

(57) ABSTRACT

The present invention relates to methods and compositions useful for treating, preventing and/or delaying the onset and/or development of schizophrenia by administering a hydrogenated pyrido[4,3-b]indole, such as dimebon, or a pharmaceutically acceptable salt thereof, to an individual.

### CROSS-REFERENCE TO RELATED APPLICATIONS

This application claims priority under 35 U.S.C. § 119 to Russian Patent Application No. 2006101999, filed with the Russian Patent Office on Jan. 25, 2006, which is incorporated herein by reference in its entirety.

### STATEMENT REGARDING FEDERALLY SPONSORED RESEARCH OR DEVELOPMENT

Not applicable.

### BACKGROUND OF THE INVENTION

Summary of Schizophrenia

Schizophrenia dramatically affects the health and well-being of individuals who suffer from this mental disorder, which is among the most severe and difficult to treat. Individuals with schizophrenia (“schizophrenics”) can suffer from a myriad of symptoms and may require significant custodial care and continuous drug and/or behavior therapy, leading to substantial social and economic costs, even in the absence of hospitalization or institutionalization. Schizophrenia affects approximately 2 million Americans. The illness usually develops between adolescence and age 30 and is characterized by one or more positive symptoms (e.g., delusions and hallucinations) and/or negative symptoms (e.g., blunted emotions and lack of interest) and/or disorganized symptoms (e.g., confused thinking and speech or disorganized behavior and perception). Schizophrenics have been demonstrated in many studies to have degraded abilities at tasks requiring short-term verbal working memory, rapidly associated cognitive “prediction” or “expectation”, or ongoing attention/vigilance control. Schizophrenics who have auditory hallucinations (which describes the majority of afflicted individuals) also have a strongly correlated degradation in their speech reception abilities. Schizophrenics also have social and functional skill deficits, e.g., deficits and confusion in identifying the moods or reactions of others, in determining what for them is a socially correct course of action and in identifying the sources of current and past actions or events. Schizophrenia is a chronic disorder and most patients require constant treatment to alleviate or decrease the incidence of psychotic episodes. The causes of schizophrenia are largely unknown. Although it is believed to have a genetic component, environmental factors appear to influence the onset and severity of the disease.

Summary of Mechanistic Considerations in the Pathogenesis of Schizophrenia

Until recently, the attention of researchers working in the field of the biochemistry of psychoses was mainly concentrated on two mediator systems: the dopamine system and the serotonin system.

The dopamine hypothesis originated from the common ability of traditional (typical) antipsychotic drugs to cause neurological side effects similar to the symptoms of Parkinson's disease. This same property also gave the drugs the common name neuroleptics. The neurobiochemistry of parkinsonism is connected with disruption of the balance between the dopamine and cholinergic systems in the nigrostriatum, in which the activity of the dopamine structures decreases, while the activity of the cholinergic structures increases. The ability of typical neuroleptics to control productive symptomatology in patients suffering from schizophrenic disorder (delusions, hallucinations, behavioral confusion) correlates with the ability to cause parkinsonism and results from the property of suppressing the activity of the dopamine system. Thus, it was concluded that positive symptomatology of a psychosis is due to excessive activity of the dopaminergic system. One more argument in favor of this finding was the result of investigating dopamine metabolites in the spinal fluid. Higher levels of homovanilic acid (a product of dopamine metabolism) were found in psychotic patients than in healthy people. Currently this hypothesis has been developed further under the influence of new data involving the results of post-mortem examinations of the brain and positron emission tomography of living patients. The important regulator role of dopamine receptors was revealed by close study of the changes of function of the dopaminergic system under the effect of neuroleptic drugs. Several types of dopamine receptors have been described, each of which has its own features of localization and function.

The second hypothesis assumes that the fundamental cause is disruption in the relationship between the dopamine and serotonin systems. The serotoninergic structures carry out a complex modulating effect on the function of the dopaminergic system by increasing its activity in the mesolimbic and mesostriatal structures and reducing it in the prefrontal region, conditioning clinical hypofrontal function phenomena. A weighty argument for this hypothesis is usually considered to be the introduction of the prototype of atypical antipsychotics, clozapine, into clinical practice. The neurochemical spectrum of activity of clozapine distinguished it from all of the neuroleptics known at that time, since clozapine blocked serotoninergic receptors substantially more strongly than dopaminergic receptors. In addition, it proved to be effective with respect to illnesses where primary deficit disorders predominated and also in most cases that exhibited resistance to traditional neuroleptics. Moreover, clozapine caused neuroleptic side effects significantly less often. J. M. Kane, “The new antipsychotics,” J. Pract. Psychiatry Behav. Health, 1997, 3:343-354.

The hypotheses described above have sufficient explanatory power with respect to a large body of facts. However, not all data fit into them. It is known that the blockade of dopaminergic receptors occurs much faster than the clinical effect develops. In addition, the degree of blockade of these receptors is the same in patients who react well to antipsychotic therapy and patients who are resistant to it (S. Heckers, “Neural models of schizophrenia,” Dialogues in Clinical Neuroscience, 2000, 2(3): 267-280). On the other hand, the attempts of psychopharmacologists to develop a drug with antipsychotic effects that does not affect the dopaminergic system still have not led to success (S. Kapur, G. Remington, “Dopamine D(2) receptors and their role in atypical antipsychotic action: still necessary and may even be sufficient,” Biol. Psychiatry, 2001, 50 (11):873-83).

Besides the widely recognized importance of the dopamine and serotonin activity of antipsychotic agents for the realization of their clinical activity, one more neuromediator system draws attention to itself. This is the glutamatergic neuromediator system of the central nervous system (CNS). Since many researchers in recent years have tended toward the opinion that cognitive disruptions play a fundamental role in the formation of schizophrenic disorder (N. C. Andreasen, “Schizophrenia: the fundamental questions,” Brain Res. Rev., 2000, 31(2-3):106-12), the glutamatergic system is causing ever growing interest, not only theoretically, but also practically (K. Hashimoto, M. Iyo, “Glutamate hypothesis of schizophrenia and targets for new antipsychotic drugs,” Nihon Shinkei Seishin Yakurigaku Zasshi, 2002, 22 (1):3-13). Stimulation of glutamatergic transmission can lead to stimulation of the activity of the central nervous system, but at some point it can also lead to toxic effects for the brain. On the other hand, depression of the glutamatergic system can lead to neuroprotector effects, but along with them, to a cognitive deficit (S. Heckers, C. Konradi, “Hippocampal neurons in schizophrenia,” J. Neural Transm., 2002, 109(5-6):891-905). Some researchers are proposing the ability to produce a glutamatergic effect as one possible neurochemical mechanism of the antideficit activity of clozapine (L. Chen, C. R. Yang, “Interaction of dopamine D1 and NMDA receptors mediates acute clozapine potentiation of glutamate EPSPs in rat prefrontal cortex,” J. Neurophysiol, 2002, 87 (5):2324-36). In addition, the glutamatergic system is ascribed the role of coordination of the functioning of other mediator structures of the brain. This function can be implemented, in particular, due to the hypothetical ability of the cerebellum (in the functioning of which the glutamergic system plays an important role) to form temporary organization of mental processes (N. C. Andreasen, “Schizophrenia: the fundamental questions,” Brain Res. Rev. 2000, 31 (2-3):106-12). Control of this function is hardly achievable for traditional antipsychotic drugs. However, the glutamate activity of clozapine in this connection yields an opportunity for the formation of new hypotheses that explain its unusual clinical activity over a long course of treatment (L. Chen, C. R. Yang, “Interaction of dopamine D1 and NMDA receptors mediates acute clozapine potentiation of glutamate EPSPs in rat prefrontal cortex,” J. Neurophysiol, 2002; 87(5): 2324-36), and the formation of new homeostatic relationships requiring a long period of time. In spite of the instantaneous blockade of dopamine receptors, the first signs of the clinical effect of antipsychotics (control of productive symptoms) are realized gradually, over several weeks, and the improvement of the patient's conditions lasts many months.

Thus, along with the theory of the pathogenesis of schizophrenia that was developed a relatively long while ago and that is widely accepted, where the main role is given to hyperfunctioning of the dopaminergic neuromediator system of the CNS and also to imbalance in the serotoninergic mediator system, very recently there has been intensive development of a theory of pathogenesis where the main role in the development of this disease is played by disruptions in the glutamatergic neuromediator system of the CNS. It is proposed that many elements of psychic disorder that are observed in schizophrenia patients are connected with hypofunctioning of the glutamatergic system. Support for the glutamate theory of schizophrenia include the fact that phencyclidine, a blocker of the NMDA receptor ion channel, one of the principal subtypes of glutamate receptors, causes a complex of behavioral symptoms that are very similar to the behavior of schizophrenia patients in healthy volunteers: they exhibit alienation, autism, negative mood; they become unable to solve cognition problems (tests); they grow eccentric and their speech and thinking become impoverished. Currently, the phencyclidine model of schizophrenia is considered to be the closest and most adequate to the behavior of schizophrenia patients (R. M. Allen, S. J. Young, “Phencyclidine-induced psychosis,” Amer. J. Psychiatry, 1976, 33:1425-8). Similar effects are also caused by other NMDA receptor ion channel blockers such as ketamine and MK-801. It has been shown that schizophrenia patients exhibit a lower level of glutaminic acid in the cerebrospinal fluid than normal people. It has also been shown in subsequent studies that the brain of schizophrenia patients shows an increase of large diameter glutamatergic fibers that is 30% over that in the brain of patients not suffering from schizophrenia and that there is a simultaneous decrease of small diameter glutamatergic fibers by 78%. In addition, an increase of the number of NMDA receptors is seen in the cerebral cortex in schizophrenia patients, but there is also a decrease of the reverse capture of glutamate in basal ganglia.

In accordance with the dopamine theory of schizophrenia, dopaminergic substances, firstly D2 subtype dopamine receptor blockers such as in particular haloperidol, aminazine, clozapine and many others, are widely used to treat patients. They efficiently alleviate the phase of acute psychosis in schizophrenia patients, but frequently prove to be much less effective in the treatment of other phases of this disease. Current therapies can also cause unpleasant side-effects and lead to difficulties in maintaining patient compliance. For this reason in recent years there has been intensive research into the mechanism of the pathogenesis of schizophrenia and the development of new drugs for effective treatment of this disease.

Summary of Hydrogenated Pyrido[4,3-b]Indole Derivatives

Known compounds of the class of tetra- and hexahydro-1H-pyrido[4,3-b]indole derivatives manifest a broad spectrum of biological activity. In the series of 2,3,4,5-tetrahydro-1H-pyrido[4,3-b]indoles the following types of activity have been found: antihistamine activity (DE 1,813,229, filed Dec. 6, 1968; DE 1,952,800, filed Oct. 20, 1969), central depressive and anti-inflammatory activity (U.S. Pat. No. 3,718,657, filed Dec. 3, 1970), neuroleptic activity (Herbert C. A., Plattner S. S., Welch W. M., Mol. Pharm. 1980, v. 17, N 1, p. 38-42) and others. 2,3,4,4a,5,9b-hexahydro-1H-pyrido[4,3-b]indole derivatives show psychotropic (Welch W. M., Harbert C. A., Weissman A., Koe B. K., J. Med. Chem., 1986, vol. 29, No. 10, p. 2093-2099), antiaggressive, antiarrhythmic and other types of activity.

Several drugs, such as diazoline (mebhydroline), dimebon, dorastine, carbidine (dicarbine), stobadine and gevotroline, based on tetra- or hexahydro-1H-pyrido[4,3-b]indole derivatives are known to have been manufactured. Diazoline (2-methyl-5-benzyl-2,3,4,5-tetrahydro-1H-pyrido[4,3-b]indole dihydrochloride) (Klyuev M. A., Drugs, used in “Medical Pract.”, USSR, Moscow, “Meditzina” Publishers, 1991, p. 512) and dimebon (2,8-dimethyl-5-(2-(6-methyl-3-pyridyl)ethyl-2,3,4,5-tetrahydro-1H-pyrido[4,3-b]indole dihydrochloride) (M. D. Mashkovsky, “Medicinal Drugs” in 2 vol. Vol. 1, 12th Edition, Moscow, “Meditzina” Publishers, 1993, p. 383) as well as dorastine (2-methyl-8-chloro-5-[2-(6-methyl-3-pyridyl)ethyl]-2,3,4,5-tetrahydro-1H-pyrido[4,3-b]indole dihydrochloride) (USAN and USP dictionary of drugs names (United States Adopted Names, 1961-1988, current US Pharmacopoeia and National Formula for Drugs and other nonproprietary drug names), 1989, 26th Edition., p. 196) are known as antihistamine drugs; carbidine (dicarbine) (cis(±)-2,8-dimethyl-2,3,4,4a,5,9b-hexahydro-1H-pyrido[4,3-b]indole dihydrochloride) is a neuroleptic agent having an antidepressive effect (L. N. Yakhontov, R. G. Glushkov, Synthetic Drugs, ed. by A. G. Natradze, Moscow, “Meditzina” Publishers, 1983, p. 234-237), and its (−)isomer, stobadine, is known as an antiarrythmic agent (Kitlova M., Gibela P., Drimal J., Bratisl. Lek. Listy, 1985, vol. 84, No. 5, p. 542-549); gevotroline 8-fluoro-2-(3-(3-pyridyl)propyl)-2,3,4,5-tetrahydro-1H-pyrido[4,3-b]indole dihydrochloride is an antipsychotic and anxiolytic agent (Abou-Gharbi M., Patel U. R., Webb M. B., Moyer J. A., Ardnee T. H., J. Med. Chem., 1987, vol. 30, p. 1818-1823). Dimebon has been used in medicine as an antiallergic agent (Inventor's Certificate No. 1138164, IP Class A61K 31/47,5, C07 D 209/52, published on Feb. 7, 1985) in Russia for over 20 years.

As described in U.S. Pat. Nos. 6,187,785 and 7,021,206, hydrogenated pyrido[4,3-b]indole derivatives, such as dimebon, have NMDA antagonist properties, which make them useful for treating neurodegenerative diseases, such as Alzheimer's disease. As described in WO 2005/055951, hydrogenated pyrido[4,3-b]indole derivatives, such as dimebon, are useful as human or veterinary geroprotectors e.g., by delaying the onset and/or development of an age-associated or related manifestation and/or pathology or condition, including disturbance in skin-hair integument, vision disturbance and weight loss. U.S. patent application Ser. Nos. 11/543,529 and 11/543,341 disclose hydrogenated pyrido[4,3-b]indole derivatives, such as dimebon, as neuroprotectors for use in treating and/or preventing and/or slowing the progression or onset and/or development of Huntington's disease.

### Significant Medical Need

There remains a significant interest in and need for additional or alternative therapies for treating, preventing and/or delaying the onset and/or development of schizophrenia. Preferably, the therapeutic agents can improve the quality of life for patients with schizophrenia.

### BRIEF SUMMARY OF THE INVENTION

Methods, compounds and compositions for treating and/or preventing and/or delaying the onset and/or the development of schizophrenia using a hydrogenated [4,3-b]indole or pharmaceutically acceptable salt thereof are described. The methods and compositions may comprise the compounds detailed herein, including without limitation the compound dimebon (2,8-dimethyl-5-(2-(6-methyl-3-pyridyl)ethyl)-2,3,4,5-tetrahydro-1H-pyrido[4,3-b]indole dihydrochloride).

In one variation, the invention embraces a method of: (a) treating schizophrenia in an individual in need thereof; (b) slowing the progression of schizophrenia in an individual who has been diagnosed with schizophrenia; or (c) preventing or delaying development of schizophrenia in an individual who is at risk of developing schizophrenia, the method comprising administering to the individual an effective amount of a hydrogenated pyrido[4,3-b]indole or pharmaceutically acceptable salt thereof, wherein the hydrogenated pyrido[4,3-b]indole is not stobadine or flutroline and does not comprise the moiety 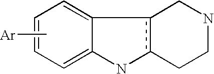
where the bond indicated by the dotted line may be a single or a double bond and the moiety is optionally substituted (meaning that where no atom or bond is indicated, the position may be filled by one or more atom (e.g., H) or other organic or inorganic moiety (e.g., —CH₃) and Ar indicates an aryl moiety. In one variation, the method is a method of alleviating one or more positive symptoms of schizophrenia by administering to an individual an effective amount of a hydrogenated pyrido[4,3-b]indole or pharmaceutically acceptable salt thereof. In one variation, the method is a method of alleviating one or more negative symptoms of schizophrenia by administering to an individual an effective amount of a hydrogenated pyrido[4,3-b]indole or pharmaceutically acceptable salt thereof. In one variation, the method is a method of alleviating one or more disorganized symptoms of schizophrenia by administering to an individual an effective amount of a hydrogenated pyrido[4,3-b]indole or pharmaceutically acceptable salt thereof. In any method or other embodiment described herein, the hydrogenated pyrido[4,3-b]indole or pharmaceutically acceptable salt thereof may exclude stobadine or flutroline and those compounds that comprise the moiety 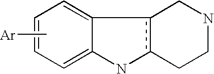
where the bond indicated by the dotted line may be a single or a double bond and the moiety is optionally substituted.

### DETAILED DESCRIPTION OF THE INVENTION

For use herein, unless clearly indicated otherwise, use of the terms “a”, “an” and the like refers to one or more.

It is also understood and clearly conveyed by this disclosure that reference to “the compound” or “a compound” includes and refers to any compound or pharmaceutically acceptable salt or other form thereof as described herein, such as the compound dimebon.

As used herein, the term “schizophrenia” includes all forms and classifications of schizophrenia known in the art, including, but not limited to catatonic type, hebephrenic type, disorganized type, paranoid type, residual type or undifferentiated type schizophrenia and deficit syndrome and/or those described in American Psychiatric Association: Diagnostic and Statistical Manual of Mental Disorders, Fourth Edition, Washington D.C., 2000 or in International Statistical Classification of Diseases and Related Health Problems, or otherwise known to those of skill in the art.

As used herein, “treatment” or “treating” is an approach for obtaining a beneficial or desired result, including clinical results. For purposes of this invention, beneficial or desired results include, but are not limited to, alleviation of symptoms associated with schizophrenia, diminishment of the extent of the symptoms associated with schizophrenia, preventing a worsening of the symptoms associated with schizophrenia, including positive and/or negative and/or disorganized symptoms. Preferably, treatment with a compound disclosed herein, such as dimebon, is accompanied by no or fewer side effects than those that are commonly associated with administration of anti-psychotic drugs, such as extrapyramidal side effects (EPS), acute dystonia, acute dyskinesia, and tardive dyskinesia.

For use herein, unless clearly indicated otherwise, “an individual” as used herein intends a mammal, including but not limited to a human. The individual may be a human who has been diagnosed with or is suspected of having or is at risk of developing schizophrenia. The individual may be a human who exhibits one or more symptoms associated with schizophrenia. The individual may be a human who is genetically or otherwise predisposed to developing schizophrenia.

For use herein, unless clearly indicated otherwise, the compounds may be administered to the individual by any available dosage form. In one variation, the compound is administered to the individual as a conventional immediate release dosage form. In one variation, the compound is administered to the individual as a sustained release form or part of a sustained release system, such as a system capable of sustaining the rate of delivery of a compound to an individual for a desired duration, which may be an extended duration such as a duration that is longer than the time required for a corresponding immediate-release dosage form to release the same amount (e.g., by weight or by moles) of compound, and can be hours or days. A desired duration may be at least the drug elimination half life of the administered compound and may be, e.g., at least about 6 hours or at least about 12 hours or at least about 24 hours or at least about 30 hours or at least about 48 hours or at least about 72 hours or at least about 96 hours or at least about 120 hours or at least about 144 or more hours, and can be at least about one week, at least about 2 weeks, at least about 3 weeks, at least about 4 weeks, at least about 8 weeks, or at least about 16 weeks or more.

The term “effective amount” intends such amount of a compound described herein such as a compound described by the Formula (1) or (2) or (A) or (B), which in combination with its parameters of efficacy and toxicity, as well as based on the knowledge of the practicing specialist should be effective in a given therapeutic form. As is understood in the art, an effective amount may be in one or more doses.

The compound may be formulated with suitable carriers for any available delivery route, whether in immediate or sustained release form, including oral, mucosal (e.g., nasal, sublingual, vaginal, buccal or rectal), parenteral (e.g., intramuscular, subcutaneous, or intravenous), topical or transdermal delivery. A compound may be formulated with suitable carriers to provide delivery forms, which may be but are not required to be sustained release forms, that include, but are not limited to: tablets, caplets, capsules (such as hard gelatin capsules and soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

The amount of compound such as dimebon in a delivery form may be any effective amount, which may be from about 10 ng to about 1,500 mg or more. In one variation, a delivery form, such as a sustained release system, comprises less than about 30 mg of compound. In one variation, a delivery form, such as a single sustained release system capable of multi-day administration, comprises an amount of compound such that the daily dose of compound is less than about 30 mg of compound.

A treatment regimen involving a dosage form of compound, whether immediate release or a sustained release system, may involve administering the compound to the individual in dose of between about 0.1 and about 10 mg/kg of body weight, at least once a day and during the period of time required to achieve the therapeutic effect. In other variations, the daily dose (or other dosage frequency) of a hydrogenated pyrido[4,3-b]indole as described herein is between about 0.1 and about 8 mg/kg; or between about 0.1 to about 6 mg/kg; or between about 0.1 and about 4 mg/kg; or between about 0.1 and about 2 mg/kg; or between about 0.1 and about 1 mg/kg; or between about 0.5 and about 10 mg/kg; or between about 1 and about 10 mg/kg; or between about 2 and about 10 mg/kg; or between about 4 to about 10 mg/kg; or between about 6 to about 10 mg/kg; or between about 8 to about 10 mg/kg; or between about 0.1 and about 5 mg/kg; or between about 0.1 and about 4 mg/kg; or between about 0.5 and about 5 mg/kg; or between about 1 and about 5 mg/kg; or between about 1 and about 4 mg/kg; or between about 2 and about 4 mg/kg; or between about 1 and about 3 mg/kg; or between about 1.5 and about 3 mg/kg; or between about 2 and about 3 mg/kg; or between about 0.01 and about 10 mg/kg; or between about 0.01 and 4 mg/kg; or between about 0.01 mg/kg and 2 mg/kg; or between about 0.05 and 10 mg/kg; or between about 0.05 and 8 mg/kg; or between about 0.05 and 4 mg/kg; or between about 0.05 and 4 mg/kg; or between about 0.05 and about 3 mg/kg; or between about 10 kg to about 50 kg; or between about 10 to about 100 mg/kg or between about 10 to about 250 mg/kg; or between about 50 to about 100 mg/kg or between about 50 and 200 mg/kg; or between about 100 and about 200 mg/kg or between about 200 and about 500 mg/kg; or a dosage over about 100 mg/kg; or a dosage over about 500 mg/kg. In some embodiments, a daily dosage of dimebon is administered, such as a daily dosage that is less than about 0.1 mg/kg, which may include but is not limited to, a daily dosage of about 0.05 mg/kg.

The compound, such as dimebon, may be administered to an individual in accordance with an effective dosing regimen for a desired period of time or duration, such as at least about one month, at least about 2 months, at least about 3 months, at least about 6 months, or at least about 12 months or longer. In one variation, the compound is administered on a daily or intermittent schedule for the duration of the individual's life.

The dosing frequency can be about a once weekly dosing. The dosing frequency can be about a once daily dosing. The dosing frequency can be more than about once weekly dosing. The dosing frequency can be less than three times a day dosing. The dosing frequency can be less than about three times a day dosing. The dosing frequency can be about three times a week dosing. The dosing frequency can be about a four times a week dosing. The dosing frequency can be about a two times a week dosing. The dosing frequency can be more than about once weekly dosing but less than about daily dosing. The dosing frequency can be about a once monthly dosing. The dosing frequency can be about a twice weekly dosing. The dosing frequency can be more than about once monthly dosing but less than about once weekly dosing. The dosing frequency can be intermittent (e.g., once daily dosing for 7 days followed by no doses for 7 days, repeated for any 14 day time period, such as about 2 months, about 4 months, about 6 months or more). The dosing frequency can be continuous (e.g., once weekly dosing for continuous weeks). Any of the dosing frequencies can employ any of the compounds described herein together with any of the dosages described herein, for example, the dosing frequency can be a once daily dosage of less than 0.1 mg/kg or less than about 0.05 mg/kg of dimebon.

Methods for Treating Schizophrenia

The hydrogenated pyrido[4,3-b]indoles described herein may be used to treat and/or prevent and/or delay the onset and/or the development of schizophrenia. As illustrated in Example 1, the representative hydrogenated pyrido[4,3-b]indole dimebon is capable of reducing the blocking effect of MK-801 on NMDA-induced currents in cultured rat hippocampus neurons. Exemplary methods for determining the ability of hydrogenated pyrido[4,3-b]indoles to treat and/or prevent and/or delay the onset and/or the development of schizophrenia are described in Examples 2 and 3.

It was surprisingly found that compounds described herein, although they may be NMDA receptor blockers, may also be capable of reducing the blocking activity of MK-801 on NMDA receptors. Since it was found that phencyclidine and MK-801 act in accordance with the same mechanism, by competing for the same intrachannel segment of the NMDA receptor it should be expected that the compounds described herein will weaken the blocking effect of phencyclidine on the NMDA receptor in exactly the same way. Since the psychotomimetic properties of phencyclidine are due to its ability to stably bind to a specific segment within the NMDA receptor ion channel and to block ion currents passing through its ion channel, then the attenuation of this blocking effect by compounds described herein, such as those of Formula (1), (2), (A) or (B) should lead to a decrease of the psychotomimetic properties of phencyclidine.

Thus, the present invention provides a variety of methods, such as those described in the “Brief Summary of the Invention” and elsewhere in this disclosure. The methods of the invention employ the compounds described herein. For example, in one embodiment, the present invention provides a method of treating schizophrenia in a patient in need thereof comprising administering to the individual an effective amount of a hydrogenated pyrido[4,3-b]indole, such as dimebon or pharmaceutically acceptable salt thereof. In one embodiment, the present invention provides a method of delaying the onset and/or development of schizophrenia in an individual who is considered at risk for developing schizophrenia (e.g., an individual whose one or more family members have had schizophrenia or an individual who has been diagnosed as having a genetic mutation associated with schizophrenia or an individual who exhibits behavior consistent with the onset of schizophrenia) comprising administering to the individual an effective amount of a hydrogenated pyrido[4,3-b]indole, such as dimebon or pharmaceutically acceptable salt thereof. In one embodiment, the present invention provides a method of delaying the onset and/or development of schizophrenia in an individual who is genetically predisposed to developing schizophrenia comprising administering to the individual an effective amount of a hydrogenated pyrido[4,3-b]indole, such as dimebon or pharmaceutically acceptable salt thereof. In one embodiment, the present invention provides a method of delaying the onset and/or development schizophrenia in an individual having a mutated or abnormal gene associated with schizophrenia (such as the NRG1 or DTNBP1 gene) but who has not been diagnosed with schizophrenia comprising administering to the individual an effective amount of a hydrogenated pyrido[4,3-b]indole, such as dimebon or pharmaceutically acceptable salt thereof. In one embodiment, the present invention provides a method of preventing schizophrenia in an individual who is genetically predisposed to developing schizophrenia or who has a mutated or abnormal gene associated with schizophrenia but who has not been diagnosed with schizophrenia comprising administering to the individual an effective amount of a hydrogenated pyrido[4,3-b]indole, such as dimebon or pharmaceutically acceptable salt thereof. In one embodiment, the present invention provides a method of preventing the onset and/or development of schizophrenia in an individual who is not identified as genetically predisposed to developing schizophrenia comprising administering to the individual an effective amount of a hydrogenated pyrido[4,3-b]indole, such as dimebon or pharmaceutically acceptable salt thereof. In one embodiment, the present invention provides a method of decreasing the intensity or severity of the symptoms of schizophrenia in an individual who is diagnosed with schizophrenia comprising administering to the individual an effective amount of a hydrogenated pyrido[4,3-b]indole, such as dimebon or pharmaceutically acceptable salt thereof. In one embodiment, the present invention provides a method of enhancing the quality of life of an individual diagnosed with schizophrenia comprising administering to the individual an effective amount of a hydrogenated pyrido[4,3-b]indole, such as dimebon or pharmaceutically acceptable salt thereof. In one variation, the method comprises the manufacture of a medicament for use in any of the above methods, e.g., treating and/or preventing and/or delaying the onset or development of schizophrenia.

Compounds for Use in the Methods, Formulations, Kits and Inventions Discloses Herein

When reference to organic residues or moieties having a specific number of carbons is made, unless clearly stated otherwise, it intends all geometric and other isomers thereof. For example, “butyl” includes n-butyl, sec-butyl, isobutyl and t-butyl; “propyl” includes n-propyl and isopropyl.

The term “alkyl” intends and includes linear, branched or cyclic hydrocarbon structures and combinations thereof. Preferred alkyl groups are those having 20 carbon atoms (C20) or fewer. More preferred alkyl groups are those having fewer than 15 or fewer than 10 or fewer than 8 carbon atoms.

The term “lower alkyl” refers to alkyl groups of from 1 to 5 carbon atoms. Examples of lower alkyl groups include methyl, ethyl, propyl, isopropyl, butyl, s- and t-butyl and the like. Lower alkyl is a subset of alkyl.

The term “aryl” or (“Ar”) refers to an unsaturated aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxain-3(4H)-one-7-yl), and the like. Preferred aryls includes phenyl and naphthyl.

The term “heteroaryl” refers to an aromatic carbocyclic group of from 2 to 10 carbon atoms and 1 to 4 heteroatoms selected from oxygen, nitrogen and sulfur within the ring. Such heteroaryl groups can have a single ring (e.g., pyridyl or furyl) or multiple condensed rings (e.g., indolizinyl or benzothienyl). Examples of heteroaryl residues include, e.g., imidazolyl, pyridinyl, indolyl, thiopheneyl, thiazolyl, furanyl, benzimidazolyl, quinolinyl, isoquinolinyl, pyrimidinyl, pyrazinyl, tetrazolyl and pyrazolyl.

The term “aralkyl” refers to a residue in which an aryl moiety is attached to the parent structure via an alkyl residue. Examples are benzyl, phenethyl and the like.

The term “heteroaralkyl” refers to a residue in which a heteroaryl moiety is attached to the parent structure via an alkyl residue. Examples include furanylmethyl, pyridinylmethyl, pyrimidinylethyl and the like.

The term “substituted heteroaralkyl” refers to heteroaryl groups which are substituted with from 1 to 3 substituents, such as residues selected from the group consisting of hydroxy, alkyl, alkoxy, alkenyl, alkynyl, amino, aryl, carboxyl, halo, nitro and amino.

The term “halo” or “halogen” refers to fluoro, chloro, bromo and iodo.

Compounds for use in the systems, methods and kits described herein are hydrogenated pyrido[4,3-b]indoles or pharmaceutically acceptable salts thereof, such as an acid or base salt thereof. A hydrogenated pyrido[4,3-b]indole can be a tetrahydro pyrido[4,3-b]indole or pharmaceutically acceptable salt thereof. The hydrogenated pyrido[4,3-b]indole can also be a hexahydro pyrido[4,3-b]indole or pharmaceutically acceptable salt thereof. The hydrogenated pyrido[4,3-b]indole compounds can be substituted with 1 to 3 substituents, although unsubstituted hydrogenated pyrido[4,3-b]indole compounds or hydrogenated pyrido[4,3-b]indole compounds with more than 3 substituents are also contemplated. Suitable substituents include but are not limited to alkyl, lower alkyl, aralkyl, heteroaralkyl, substituted heteroaralkyl, and halo.

Particular hydrogenated pyrido[4,3-b]indoles are exemplified by the Formulae A and B: 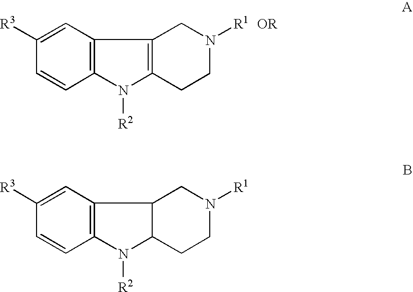
where R¹ is selected from the group consisting of alkyl, lower alkyl and aralkyl, R² is selected from the group consisting of hydrogen, aralkyl and substituted heteroaralkyl; and R³ is selected from the group consisting of hydrogen, alkyl, lower alkyl and halo.

In one variation, R¹ is alkyl, such as an alkyl selected from the group consisting of C₁-C₁₅alkyl, C₁₀-C₁₅alkyl, C₁-C₁₀alkyl, C₂-C₁₅alkyl, C₂-C₁₀alkyl, C₂-C₈alkyl, C₄-C₈alkyl, C₆-C₈alkyl, C₆-C₁₅alkyl, C₁₅-C₂₀alkyl; C₁-C₈alkyl and C₁-C₆alkyl. In one variation, R¹ is aralkyl. In one variation, R¹ is lower alkyl, such as a lower alkyl selected from the group consisting of C₁-C₂alkyl, C₁-C₄alkyl, C₂-C₄ alkyl, C₁-C₅ alkyl, C₁-C₃alkyl, and C₂-C₅alkyl.

In one variation, R¹ is a straight chain alkyl group. In one variation, R¹ is a branched alkyl group. In one variation, R¹ is a cyclic alkyl group.

In one variation, R¹ is methyl. In one variation, R¹ is ethyl. In one variation, R¹ is methyl or ethyl. In one variation, R¹ is methyl or an aralkyl group such as benzyl. In one variation, R¹ is ethyl or an aralkyl group such as benzyl.

In one variation, R¹ is an aralkyl group. In one variation, R¹ is an aralkyl group where any one of the alkyl or lower alkyl substituents listed in the preceding paragraphs is further substituted with an aryl group (e.g., Ar—C₁-C₆alkyl, Ar—C₁-C₃alkyl or Ar—C₁-C₁₅alkyl). In one variation, R¹ is an aralkyl group where any one of the alkyl or lower alkyl substituents listed in the preceding paragraphs is substituted with a single ring aryl residue. In one variation, R¹ is an aralkyl group where any one of the alkyl or lower alkyl substituents listed in the preceding paragraphs is further substituted with a phenyl group (e.g., Ph-C₁-C₆Alkyl or Ph-C₁-C₃Alkyl, Ph-C₁-C₁₅alkyl). In one variation, R¹ is benzyl.

All of the variations for R¹ are intended and hereby clearly described to be combined with any of the variations stated below for R² and R³ the same as if each and every combination of R¹, R² and R³ were specifically and individually listed.

In one variation, R² is H. In one variation, R² is an aralkyl group. In one variation, R² is a substituted heteroaralkyl group. In one variation, R² is hydrogen or an aralkyl group. In one variation, R² is hydrogen or a substituted heteroaralkyl group. In one variation, R² is an aralkyl group or a substituted heteroaralkyl group. In one variation, R² is selected from the group consisting of hydrogen, an aralkyl group and a substituted heteroaralkyl group.

In one variation, R² is an aralkyl group where R² can be any one of the aralkyl groups noted for R¹ above, the same as if each and every aralkyl variation listed for R¹ is separately and individually listed for R².

In one variation, R² is a substituted heteroaralkyl group, where the alkyl moiety of the heteroaralkyl can be any alkyl or lower alkyl group, such as those listed above for R¹. In one variation, R² is a substituted heteroaralkyl where the heteroaryl group is substituted with 1 to 3 C₁-C₃ alkyl substituents (e.g., 6-methyl-3-pyridylethyl). In one variation, R² is a substituted heteroaralkyl group wherein the heteroaryl group is substituted with 1 to 3 methyl groups. In one variation, R² is a substituted heteroaralkyl group wherein the heteroaryl group is substituted with one lower alkyl substituent. In one variation, R² is a substituted heteroaralkyl group wherein the heteroaryl group is substituted with one C₁-C₃ alkyl substituent. In one variation, R² is a substituted heteroaralkyl group wherein the heteroaryl group is substituted with one or two methyl groups. In one variation, R² is a substituted heteroaralkyl group wherein the heteroaryl group is substituted with one methyl group.

In other variations, R² is any one of the substituted heteroaralkyl groups in the immediately preceding paragraph where the heteroaryl moiety of the heteroaralkyl group is a single ring heteroaryl group. In other variations, R² is any one of the substituted heteroaralkyl groups in the immediately preceding paragraph where the heteroaryl moiety of the heteroaralkyl group is a multiple condensed ring heteroaryl group. In other variations, R² is any one of the substituted heteroaralkyl groups in the immediately preceding paragraph where the heteroaralkyl moiety is a pyridyl group (Py).

In one variation, R² is 6-CH₃-3-Py-(CH₂)₂—.

In one variation, R³ is hydrogen. In other variations, R³ is any one of the alkyl groups noted for R¹ above, the same as if each and every alkyl variation listed for R¹ is separately and individually listed for R³. In another variation, R³ is a halo group. In one variation, R³ is hydrogen or an alkyl group. In one variation, R³ is a halo or alkyl group. In one variation, R³ is hydrogen or a halo group. In one variation, R³ is selected from the group consisting of hydrogen, alkyl and halo. In one variation, R³ is Br. In one variation, R³ is I. In one variation, R³ is F. In one variation, R³ is Cl.

In a particular variation, the hydrogenated pyrido[4,3-b]indole is 2,8-dimethyl-5-(2-(6-methyl-3-pyridyl)ethyl)-2,3,4,5-tetrahydro-1H-pyrido[4,3-b]indole or a pharmaceutically acceptable salt thereof.

The hydrogenated pyrido[4,3-b]indoles can be in the form of pharmaceutically acceptable salts thereof, which are readily known to those of skill in the art. The pharmaceutically acceptable salts include pharmaceutically acceptable acid salts. Examples of particular pharmaceutically acceptable salts include hydrochloride salts or dihydrochloride salts. In a particular variation, the hydrogenated pyrido[4,3-b]indole is a pharmaceutically acceptable salt of 2,8-dimethyl-5-(2-(6-methyl-3-pyridyl)ethyl)-2,3,4,5-tetrahydro-1H-pyrido[4,3-b]indole, such as 2,8-dimethyl-5-(2-(6-methyl-3-pyridyl)ethyl)-2,3,4,5-tetrahydro-1H-pyrido[4,3-b]indole dihydrochloride (dimebon).

Particular hydrogenated pyrido-([4,3-b]) indoles can also be described by the Formula (1) or by the Formula (2):

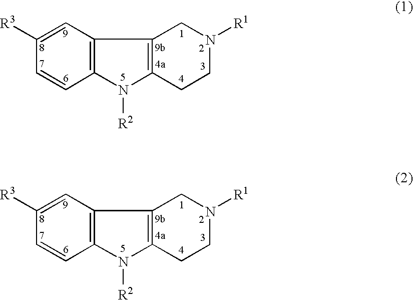

For compounds of a general Formula (1) or (2),

R¹ represents —CH₃, CH₃CH₂—, or PhCH₂— (benzyl);

R² is —H, PhCH₂—, or 6-CH₃-3-Py-(CH₂)₂—;

R³ is —H, —CH₃, or —Br,

in any combination of the above substituents. All possible combinations of the substituents of Formulae (1) and (2) are contemplated as specific and individual compounds the same as if each single and individual compound were listed by chemical name. Also contemplated are the compounds of Formula (1) or (2), with any deletion of one or more possible moieties from the substituent groups listed above: e.g., where R¹ represents —CH₃; R² is —H, PhCH₂—, or 6-CH₃-3-Py-(CH₂)₂—; and R³ is —H, —CH₃, or —Br, or where R¹ represents —CH₃; R² is 6-CH₃-3-Py-(CH₂)₂—; and R³ represents —H, —CH₃, or —Br.

The above and any compound herein may be in a form of salts with pharmaceutically acceptable acids and in a form of quaternized derivatives.

The compound may be Formula (1), where R¹ is —CH₃, R² is —H, and R³ is —CH₃. In one variation, the compound is of the Formula (1), provided that the substituents are not where R¹ is —CH₃, R²—H, and R³ is —CH₃. The compound may be Formula (2), where R¹ is represented by —CH₃, CH₃CH₂—, or PhCH₂—; R² is —H, PhCH₂—, or 6-CH₃-3-Py-(CH₂)₂—; R³ is —H, —CH₃, or —Br. The compound may be Formula (2), where R¹ is CH₃CH₂— or PhCH₂—, R² is —H, and R³ is —H; or a compound, where R¹ is —CH₃, R² is PhCH₂—, R³ is —CH₃; or a compound, where R¹ is —CH₃, R² is 6-CH₃-3-Py-(CH₂)₂—, and R³ is —CH₃; or a compound, where R¹ is —CH₃, R is —H, R³ is —H or —CH₃; or a compound, where R¹ is —CH₃, R² is —H, R³ is —Br.

Compounds known from literature which can be used in the methods disclosed herein include the following specific compounds:
1. cis(±) 2,8-dimethyl-2,3,4,4a,5,9b-hexahydro-1H-pyrido[4,3-b]indole and its dihydrochloride;
2. 2-ethyl-2,3,4,5-tetrahydro-1H-pyrido[4,3-b]indole;
3. 2-benzyl-2,3,4,5-tetrahydro-1H-pyrido[4,3-b]indole;
4. 2,8-dimethyl-5-benzyl-2,3,4,5-tetrahydro-1H-pyrido[4,3-b]indole and its dihydrochloride;
5. 2-methyl-5-(2-methyl-3-pyridyl)ethyl-2,3,4,5-tetrahydro-1H-pyrido[4,3-b]indole and its sesquisulfate;
6. 2,8-dimethyl-5-(2-(6-methyl-3-pyridyl)ethyl)-2,3,4,5-tetrahydro-1H-pyrido[4,3-b]indole and its dihydrochloride (dimebon);
7. 2-methyl-2,3,4,5-tetrahydro-1H-pyrido[4,3-b]indole;
8. 2,8-dimethyl-2,3,4,5-tetrahydro-1H-pyrido[4,3-b]indole and its methyl iodide;
9. 2-methyl-8-bromo-2,3,4,5-tetrahydro-1H-pyrido[4,3-b]indole and its hydrochloride.

In one variation, the compound is of the Formula A or B and R¹ is selected from a lower alkyl or benzyl; R² is selected from a hydrogen, benzyl or 6-CH₃-3-Py-(CH₂)₂— and R³ is selected from hydrogen, lower alkyl or halo, or any pharmaceutically acceptable salt thereof. In another variation, R¹ is selected from —CH₃, CH₃CH₂—, or benzyl; R² is selected from —H, benzyl, or 6-CH₃-3-Py-(CH₂)₂—; and R³ is selected from —H, —CH₃ or —Br, or any pharmaceutically acceptable salt thereof. In another variation the compound is selected from the group consisting of: cis(±) 2,8-dimethyl-2,3,4,4a,5,9b-hexahydro-1H-pyrido[4,3-b]indole as a racemic mixture or in the substantially pure (+) or substantially pure (−) form; 2-ethyl-2,3,4,5-tetrahydro-1H-pyrido[4,3-b]indole; 2-benzyl-2,3,4,5-tetrahydro-1H-pyrido[4,3-b]indole; 2,8-dimethyl-5-benzyl-2,3,4,5-tetrahydro-1H-pyrido[4,3-b]indole; 2-methyl-5-(2-methyl-3-pyridyl)ethyl-2,3,4,5-tetrahydro-1H-pyrido[4,3-b]indole; 2,8-dimethyl-5-(2-(6-methyl-3-pyridyl)ethyl)-2,3,4,5-tetrahydro-1H-pyrido[4,3-b]indole; 2-methyl-2,3,4,5-tetrahydro-1H-pyrido[4,3-b]indole; 2,8-dimethyl-2,3,4,5-tetrahydro-1H-pyrido[4,3-b]indole; or 2-methyl-8-bromo-2,3,4,5-tetrahydro-1H-pyrido[4,3-b]indole or any pharmaceutically acceptable salt of any of the foregoing. In one variation, the compound is of the Formula A or B wherein R¹ is —CH₃, R² is —H and R³ is —CH₃ or any pharmaceutically acceptable salt thereof. The compound may be of the Formula A or B where R¹ is CH₃CH₂— or benzyl, R² is —H, and R³ is —CH₃ or any pharmaceutically acceptable salt thereof. The compound may be of the Formula A or B where R¹ is —CH₃, R² is benzyl, and R³ is —CH₃ or any pharmaceutically acceptable salt thereof. The compound may be of the Formula A or B where R¹ is —CH₃, R² is 6-CH₃-3-Py-(CH₂)₂—, and R³ is —H or any pharmaceutically acceptable salt thereof. The compound may be of the Formula A or B where R² is 6-CH₃-3-Py-(CH₂)₂— or any pharmaceutically acceptable salt thereof. The compound may be of the Formula A or B where R¹ is —CH₃, R² is —H, and R³ is —H or —CH₃ or any pharmaceutically acceptable salt, thereof. The compound may be of the Formula A or B where R¹ is —CH₃, R² is —H, and R³ is —Br, or any pharmaceutically acceptable salt thereof. The compound may be of the Formula A or B where R¹ is selected from a lower alkyl or aralkyl, R² is selected from a hydrogen, aralkyl or substituted heteroaralkyl and R³ is selected from hydrogen, lower alkyl or halo.

The compound for use in the systems and methods may be 2,8-dimethyl-5-(2-(6-methyl-3-pyridyl)ethyl-2,3,4,5-tetrahydro-1H-pyrido[4,3-b]indole or any pharmaceutically acceptable salt thereof, such as an acid salt, a hydrochloride salt or a dihydrochloride salt thereof.

Any of the compounds disclosed herein having two stereocenters in the pyrido[4,3-b]indole ring structure (e.g., carbons 4a and 9b of compound (1)) includes compounds whose stereocenters are in a cis or a trans form. A composition may comprise such a compound in substantially pure form, such as a composition of substantially pure S,S or R,R or S,R or R,S compound. A composition of substantially pure compound means that the composition contains no more than 15% or no more than 10% or no more than 5% or no more than 3% or no more than 1% impurity of the compound in a different stereochemical form. For instance, a composition of substantially pure S,S compound means that the composition contains no more than 15% or no more than 10% or no more than 5% or no more than 3% or no more than 1% of the R,R or S,R or R,S form of the compound. A composition may contain the compound as mixtures of such stereoisomers, where the mixture may be enanteomers (e.g., S,S and R,R) or diastereomers (e.g., S,S and R,S or S,R) in equal or unequal amounts. A composition may contain the compound as a mixture of 2 or 3 or 4 such stereoisomers in any ratio of stereoisomers. Compounds disclosed herein having stereocenters other than in the pyrido[4,3-b]indole ring structure intends all stereochemical variations of such compounds, including but not limited to enantiomers and diastereomers in any ratio, and includes racemic and enantioenriched and other possible mixtures. Unless stereochemistry is explicitly indicated in a structure, the structure is intended to embrace all possible stereoisomers of the compound depicted.

Synthesis and studies on neuroleptic properties for cis(±) 2,8-dimethyl-2,3,4,4a,5,9b-hexahydro-1H-pyrido[4,3-b]indole and its dihydrochloride are reported, for instance, in the following publication: Yakhontov, L.N., Glushkov, R.G., Synthetic therapeutic drugs. A.G. Natradze, the editor, Moscow Medicina, 1983, p. 234-237. Synthesis of compounds 2, 8, and 9 noted above as known from the literature, and data on their properties as serotonin antagonists are reported in, for instance, in C. J. Cattanach, A. Cohen & B. H. Brown in J. Chem. Soc. (Ser.C) 1968, p. 1235-1243. Synthesis of the compound 3 noted above as known from the literature is reported, for instance, in the article N. P. Buu-Hoi, O. Roussel, P. Jacquignon, J. Chem. Soc., 1964, N 2, p. 708-711. N. F. Kucherova and N. K. Kochetkov (General chemistry (russ.), 1956, v. 26, p. 3149-3154) describe the synthesis of the compound 4 noted above as known from the literature. Synthesis of compounds 5 and 6 noted above as known from the literature is described in the article by A. N. Kost, M. A. Yurovskaya, T. V. MeI'nikova, in Chemistry of heterocyclic compounds, 1973, N 2, p. 207-212. The synthesis of the compound 7 noted above as known from the literature is described by U, Horlein in Chem. Ber., 1954, Bd. 87, hft 4, 463-p. 472. M. Yurovskaya and I. L. Rodionov in Chemistry of heterocyclic compounds (1981, N 8, p. 1072-1078) describe the synthesis of methyl iodide of the compound 8 above.

One or several compounds described herein can be used in the preparation of a formulation, such as a pharmaceutical formulation, by combining the compound or compounds as an active ingredient with a pharmacologically acceptable carrier, which are known in the art. Depending on the therapeutic form of the system (e.g., transdermal patch vs. oral tablet), the carrier may be in various forms. In addition, pharmaceutical preparations may contain preservatives, solubilizers, stabilizers, re-wetting agents, emulgators, sweeteners, dyes, adjusters, salts for the adjustment of osmotic pressure, buffers, coating agents or antioxidants. Preparations comprising the compound, such as dimebon, may also contain other substances which have valuable therapeutic properties. Therapeutic forms may be represented by a usual standard dose and may be prepared by a known pharmaceutical method. Suitable formulations can be found, e.g., in Remington's Pharmaceutical Sciences, Mack Publishing Company, Philadelphia, Pa., 20^{th} ed. (2000), which is incorporated herein by reference.

The invention further provides kits comprising one or more compounds as described herein. The kits may employ any of the compounds disclosed herein and instructions for use. In one variation, the kit employs dimebon. The kits may be used for any one or more of the uses described herein, and, accordingly, may contain instructions for any one or more of the stated uses (e.g., treating and/or preventing and/or delaying the onset and/or the development of schizophrenia).

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may optionally include a set of instructions, generally written instructions, although electronic storage media (e.g., magnetic diskette or optical disk) containing instructions are also acceptable, relating to the use of component(s) of the methods of the present invention (e.g., treating, preventing and/or delaying the onset and/or the development of schizophrenia. The instructions included with the kit generally include information as to the components and their administration to an individual.

The following Examples are provided to illustrate but not limit the invention.

### EXAMPLES

### Example 1

### Method of Evaluating the NMDA-Induced Current Blocking Properties of the Compounds

The drug “dimebon,” 2,8-dimethyl-5-[2-(6-methylpyridyl-3)ethyl]-2,3,4,5-tetrahydro-1H-pyrido[4,3-b]indole dihydrochloride of the Formula: 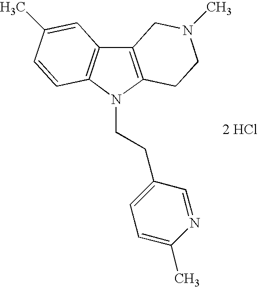
was taken as a representative of the compounds described herein.

Experiments were carried out by the patch clamp method on freshly isolated neurons of a rat brain cortex or on cultured rat hippocampus neurons. Neurons for cultivation were obtained from the hippocampus of neonatal rats (1-2 days) by the method of trypsinization followed by pipetting. Cells suspended in culture medium were placed in 3 mL quantities into the wells of a 6-well planchette (Nunc) or into Petri dishes, in which glasses coated with poly-L-lysine had first been placed. The cell concentration as a rule was 2.5×10−6−5×10−6 cell/mL. The culture medium consisted of Eagle's minimum medium and a DME/F12 medium (1:1) supplemented with 10% calf serum, glutamine (2 mM), gentamycin (50 μg/mL), glucose (15 mM) and 20 mM KCl, with the pH brought to 7-7.4 using NaHCO₃. Planchettes containing cultures were placed in a CO₂— incubator at 37° C. and 100% humidity. Cytosine arabinoside 10-20 μL was added on the second to third day of cultivation. After 6-7 days of cultivation 1 mg/mL glucose was added to the medium, or the medium was exchanged, depending on the following experiment. The cultured hippocampus neurons were placed in a 0.4 mL working chamber. The working solution had the following composition (mM): NaCl 150.0, KCl 5.0, CaCl₂ 2.6, MgSO₄×7H₂O 2.0, HEPES 10.0, glucose 15.0, pH 7.36.

Transmembrane currents produced by application of NMDA were registered by the patch clamp electrophysiological method in the whole cell configuration. Application of substances was done by the method of rapid superfusion. Currents were registered with the aid of borosilicate microelectrodes (resistance 3.0-4.5 mOhm) filled with the following composition (mM): KCl 100.0, EGTA 11.0, CaCl₂ 1.0, MgCl₂ 1.0, HEPES 10.0, ATP 5.0 pH 7.2. An EPC-9 instrument (HEKA, Germany) was used for registration. Currents were recorded on the hard disk of a Pentium-IV PC using the pulse program, which is also purchased from HEKA. The results were analyzed with the aid of the Pulsefit program (HEKA).

Application of NMDA induced inflow currents in the cultured hippocampus neurons. Dimebon had a blocking effect on currents caused by application of NMDA. The IC50 of dimebon varied from 6.0 to 10 μM, and was an average of 7.7±1.9 μM. MK-801 also caused blockade of NMDA-induced currents. This blockade had a clear “use dependence,” in other words magnitude of the blocking effect caused by MK-801 was dependent on the preceding effect of the agonist, i.e., NMDA: the blocking effect increases in a series of successive applications of the agonist up to some final value, which was dependent on the concentration of MK-801. 1 μm MK-801 caused blockade of NMDA-induced currents by 70±15%. Preliminary perfusion of neurons with a solution containing dimebon in a concentration of 10 μM caused a decrease of the blocking effect of MK-801 to 40±18%. For comparison the effect of the competing antagonist of the NMDA receptor D-AP5 (D-2-amino-5-phosphonovaleric acid—a selected NMDA receptor antagonist) was investigated for comparison. D-AP5 itself in a dose of 5 μm blocked the NMDA-induced currents by 60-80%. Preliminary application of D-AP5 did not decrease the blocking effect of MK-801.

The results that were obtained are given in Table 1.

TABLE 1
Effect of substances on NMDA-induced currents
in cultured rat hippocampus neurons.

| | Blockade of NMDA-induced |
|---|---|
| Substance | currents (%) |
| Dimebon | By 50-70% at 10 μM |
| MK-801 | By 70 ± 15% at 1 μM |
| Dimebon + MK-801 | By 40 ± 18% |
| D-AP5 | By 60-80% at 5 μM |
| D-AP5 + MK-801 | By 75 ± 17% |

The results indicate that dimebon, in spite of the fact that it is itself believed to be an antagonist of NMDA receptors, is capable of reducing the blocking effect of MK-801 on NMDA-induced currents in cultured rat hippocampus neurons. Although the mechanism of the blocking effect of dimebon on NMDA receptors has not yet been established, it does not have the neurotoxic effect that is characteristic for noncompeting blockers of the NMDA receptor ion channel—phencyclidine, MK-801 and ketamine. Based on these new results, it can be suggested that a reduction of the channel-blocking effect of MK-801 (and analogously phencyclidine) on NMDA receptors can lead to a decrease of their psychotomimetic effect and, therefore, to elimination of symptoms characteristic for schizophrenia.

These results indicate that dimebon, along with its previously described properties, can be used for effective treatment of schizophrenia.

### Example 2

### Use of an In Vivo Model to Determine the Ability to Compounds of the Invention to Treat Prevent and/or Delay the Onset and/or the Development of Schizophrenia

In vivo models of schizophrenia can be used to determine the ability of any of the hydrogenated pyrido[4,3-b]indoles described herein (e.g., dimebon) to treat and/or prevent and/or delay the onset and/or the development of schizophrenia.

One exemplary model for testing the activity of one or more hydrogenated pyrido[4,3-b]indoles described herein to treat and/or prevent and/or delay the onset and/or development of schizophrenia employs phencyclidene, which is chronically administered to the animal (e.g., non-primate (rat) or primate (monkey)), resulting in dysfunctions similar to those seen in schizophrenic humans. See Jentsch et al., 1997, Science 277:953-955 and Piercey et al., 1988, Life Sci. 43(4):375-385). Standard experimental protocols may be employed in this or in other animal models.

### Example 3

### Use of Human Clinical Trials to Determine the Ability of Compounds of the Invention to Treat, Prevent and/or Delay the Onset and/or the Development of Schizophrenia

If desired, any of the hydrogenated pyrido[4,3-b]indoles described herein (e.g., dimebon) can also be tested in humans to determine the ability of the compound to treat, prevent and/or delay the onset and/or the development of schizophrenia. Standard methods can be used for these clinical trials.

In one exemplary method, subjects with schizophrenia are enrolled in a tolerability, pharmacokinetics and pharmacodynamics phase I study of a hydrogenated pyrido[4,3-b]indole using standard protocols. Then a phase II, double-blind randomized controlled trial is performed to determine the efficacy of the hydrogenated pyrido[4,3-b]indole.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

All references, publications, patents, and patent applications disclosed herein are hereby incorporated by reference in their entireties.

1. A method of (a) treating schizophrenia in an individual in need thereof; (b) slowing the progression of schizophrenia in an individual who has been diagnosed with schizophrenia; or (c) preventing or delaying development of schizophrenia in an individual who is at risk of developing schizophrenia, the method comprising administering to the individual an effective amount of a hydrogenated pyrido[4,3-b]indole or pharmaceutically acceptable salt thereof, wherein the hydrogenated pyrido[4,3-b]indole is not stobadine or flutroline and does not comprise the moiety: 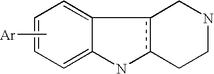
where the bond indicated by the dotted line may be a single or a double bond, Ar is an aryl group and the moiety is optionally substituted.

2. The method of claim 1, wherein the hydrogenated pyrido[4,3-b]indole is a tetrahydro pyrido[4,3-b]indole.

3. The method of claim 1, wherein the hydrogenated pyrido[4,3-b]indole is a hexahydro pyrido[4,3-b]indole.

4. The method of claim 1, wherein the hydrogenated pyrido[4,3-b]indole is of the Formula: 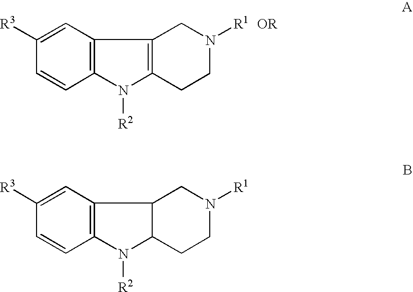
wherein:
R¹ is a lower alkyl or aralkyl;
R² is hydrogen, aralkyl or a substituted heteroaralkyl; and
R³ is hydrogen, lower alkyl or halo.

5. The method of claim 4, wherein R² is PhCH₂— or 6-CH₃-3-Py-(CH₂)₂—.

6. The method of claim 4, wherein
R¹ is CH₃—, CH₃CH₂—, or PhCH₂—;
R² is hydrogen, PhCH₂—, or 6-CH₃-3-Py-(CH₂)₂—; and
R³ is hydrogen, CH₃— or Br—.

7. The method of claim 1, wherein the hydrogenated pyrido[4,3-b]indole is selected from the group consisting of:
cis(±) 2,8-dimethyl-2,3,4,4a,5,9b-hexahydro-1H-pyrido[4,3-b]indole;
2-ethyl-2,3,4,5-tetrahydro-1H-pyrido[4,3-b]indole;
2-benzyl-2,3,4,5-tetrahydro-1H-pyrido[4,3-b]indole;
2,8-dimethyl-5-benzyl-2,3,4,5-tetrahydro-1H-pyrido[4,3-b]indole;
2-methyl-5-(2-methyl-3-pyridyl)ethyl-2,3,4,5-tetrahydro-1H-pyrido[4,3-b]indole;
2,8-dimethyl-5-(2-(6-methyl-3-pyridyl)ethyl)-2,3,4,5-tetrahydro-1H-pyrido[4,3-b]indole;
2-methyl-2,3,4,5-tetrahydro-1H-pyrido[4,3-b]indole;
2,8-dimethyl-2,3,4,5-tetrahydro-1H-pyrido[4,3-b]indole; and
2-methyl-8-bromo-2,3,4,5-tetrahydro-1H-pyrido[4,3-b]indole.

8. The method of claim 7, wherein the hydrogenated pyrido[4,3-b]indole is 2,8-dimethyl-5-(2-(6-methyl-3-pyridyl)ethyl)-2,3,4,5-tetrahydro-1H-pyrido[4,3-b]indole.

9. The method of claim 8, wherein the pharmaceutically acceptable salt is a pharmaceutically acceptable acid salt.

10. The method of claim 9, wherein the pharmaceutically acceptable salt is a hydrochloride acid salt.

11. The method of claim 1, wherein the hydrogenated pyrido[4,3-b]indole is 2,8-dimethyl-5-(2-(6-methyl-3-pyridyl)ethyl)-2,3,4,5-tetrahydro-1H-pyrido[4,3-b]indole dihydrochloride.

12. The method of claim 6, wherein R¹ is CH₃—, R² is H and R³ is CH₃—.

13. The method of claim 6, wherein R¹ CH₃CH₂— or PhCH₂—, R² is H—, and R³ is CH₃—.

14. The method of claim 6, wherein R¹ is CH₃—, R² is PhCH₂—, and R³ is CH₃—.

15. The method of claim 6, wherein R¹ is CH₃—, R² is 6-CH₃-3-Py-(CH₂)₂—, and R³ is H—.

16. The method of claim 6, where R² is 6-CH₃-3-Py-(CH₂)₂—.

17. The method of claim 6, wherein R¹ is CH₃—, R² is H—, and R³ is H— or CH₃—.

18. The method of claim 6, where R¹ is CH₃—, R² is H—, and R³ is Br—.

19. A kit comprising: (a) a hydrogenated pyrido[4,3-b]indole or pharmaceutically acceptable salt thereof and (b) instructions for use of in the treatment, prevention, slowing the progression or delaying the onset and/or development of schizophrenia.
